(19) United States
(12) Patent Application Publication
(10) Pub. No.: US 2015/0099703 A1
(43) Pub. Date: Apr. 9, 2015

(54) TREATMENT OF POSTPARTUM HAEMORRHAGE WITH CHEMICALLY MODIFIED HEPARIN OR HEPARAN SULPHATE AND A UTEROTONIC AGENT

(71) Applicant: DILAFOR AB, Solna (SE)

(72) Inventors: Gunvor Ekman-Ordeberg, Danderyd (SE); Anders Malmström, Lund (SE)

(21) Appl. No.: 14/399,450

(22) PCT Filed: May 7, 2013
(86) PCT No.: PCT/SE2013/050510

§ 371 Date: Nov. 6, 2014

Related U.S. Application Data
(60) Provisional application No. 61/644,036, filed on May 8, 2012.
(60) Provisional application No. 61/668,150, filed on Jul. 5, 2012.

(51) Int. Cl. A61K 31/727; A61K 31/48; A61K 31/5575; A61K 45/06; A61K 38/11

(52) U.S. Cl. CPC A61K 31/727; A61K 45/06; A61K 38/11; A61K 31/5575; A61K 31/48
USPC 514/11.6; 514/56; 514/54

(57) ABSTRACT

The present invention refers to the use of certain sulfated glycosaminoglycans for treatment or prevention of postpartum haemorrhage. The sulfated glycosaminoglycans have a reduced anticoagulant activity and are administered in combination with at least one uterotonic agent capable of promoting myometrial contractions of the uterus and thereby compress the vessels and cease the bleeding.

### FIELD OF INVENTION

The present invention refers to the use of certain sulfated glycosaminoglycans for the prevention and treatment of postpartum haemorrhage (PPH).

### BACKGROUND

Postpartum haemorrhage (PPH) remains a dominant factor in maternal mortality and may cause several serious complications associated with rapid blood loss. There are various definitions related to PPH, but normally it is associated with a blood loss exceeding 500-1000 ml. There are several underlying causes behind PPH and for primary haemorrhages arriving within 24 hours after delivery the most common include uterine atony, retained placenta, defects in coagulation and uterine inversion, see C W Su; Prime Care Clin Office Part, Vol. 39, 2192, pp 167-187. The clinically most frequent cause remains uterine atony which results in inadequate contraction of the myometrical fibres and insufficient occlusion of the spiral arteries leading to uncontrolled haemorrhage. The recited article by C W Su outlines a number of risk factors for acquiring uterine atony of which one is prolonged use of oxytocin as conventionally administered to induce labor or to treat labor arrest. The relationship between high levels of oxytocin and PPH is further elucidated by J Belghetti et al in British medical Journal, 2100, Vol. 1, pp 1-9.

It is a clinically accepted therapy to intervene against PPH with uterotonic agents and oxytocin is frequently given as a first hand agent to change uterine tonus, see C W Su; Prime Care Clin Office Part, Vol. 39, 2192, pp 167-187. Other agents useful to enhance uterine contractility include ergot alkaloids and prostaglandins, such as metheargine, carboprost and dinoprostone and misoprostol. There are, however, frequently situations when uterine atony is insufficiently responsive to this type of pharmacological treatments and critical surgical interventions are required.

Heparin is a naturally occurring glycosaminoglycan that is synthesized by and stored intracellulary in mast cells. The major potential adverse effects of heparin treatment are bleeding complications caused by its anticoagulant properties and low bioavailability. Heparan sulfate has glucosamine and uronic acid as repeating disaccharides and consists of N-acetylated and N-sulfated disaccharides. Heparan sulfates also possess anticoagulant activity depending on the presence of a specific anticoagulant pentasaccharide, however considerably less than heparin.

Low molecular weight heparin or depolymerized heparin is linear oligosaccharides mainly consisting of alternating N-sulfated glucosamine and IdoA residue and often containing an anticoagulant pentasaccharide. They can be prepared from heparin by specific chemical or enzymatic cleavage. Their main clinical function is to potentiate inhibition by antithrom bin of coagulation factor Xa, resulting in an antithrombotic effect. Heparin fragments having selective anticoagulant activity, as well as methods for the preparation thereof, are described in U.S. Pat. No. 4,303,651. According to the European pharmacopoeia (PharmEur) a heparin in order to be called a low molecular weight heparin (low molecular mass heparin) should have an antifactor Xa activity not less than 70 IU (International Unit)/mg and an M_{w} of less than 8 000 Da.

WO 03055499 teaches that sulfated glycosaminoglycans, such as heparin, having an anticoagulant activity of 100 BP units/mg or less, are effective for prophylactic priming or curative treatment of the cervix and the myometrium for establishing effective labor in women in general. In this document, it is suggested that sulfated glycosaminoglycans can be used in combination with oxytocin for the priming of the myometrium in cases of low endogenous oxytocin levels. It is however, not suggested that the sulfated glycosaminoglycans would be useful in directly intervening therapies when complications arise that require a direct therapeutic efficacy.

Preclinical experiments in Acta Obstetricia et Gynecologica, 2009, 88, 984-989 (Ekman-Ordeberg et al) demonstrated that pretreatment with low molecular weight heparins with or without anticoagulant activity potentiate the effect of oxytocin to contract myometrical strips obtained from cesarian sections. The authors suggest that prophylactic treatment may be a new possible therapy for protracted labor. Again it is not suggested that the low molecular weight heparins would be useful in directly intervening therapies when complications arise that require a direct therapeutic efficacy.

Consequently, there is a need to find a treatment that can manage PPH by effectively and reliably inducing myometrical contractions.

### SUMMARY OF THE INVENTION

The present invention relates to the treatment or prevention of postpartum haemorrhage (PPH), whereby a chemically modified heparin or heparan sulfate with an antifactor IIa activity of less than 10 IU/mg and an antifactor Xa activity of less than 10 IU/mg is administered in combination with an uterotonic agent capable of promoting myometrial contractions of the uterus.

### BRIEF DESCRIPTION OF THE FIGURES

FIGS. 1A-1D show calcium ion influx in uterine muscle cells when treated with combinations of oxytocin and a chemically modified heparin (DF01) as defined according to the invention.

### DESCRIPTION OF THE INVENTION

It is to be understood that the terminology employed herein is used for the purpose of describing particular embodiments only and is not intended to be limiting the invention.

It must be noted that, as used in this specification and the appended claims, the singular forms “a,” “an,” and “the” include plural referents unless the context clearly dictates otherwise.

Also, the term “about” is used to indicate a deviation of +/−2% of the given value, preferably +/−5%, and most preferably +/−10% of the numeric values, where applicable.

In one aspect of the present invention the term “postpartum haemorrhage (PPH)” is defined as a blood loss of about 500 ml or more after vaginal delivery and about 1000 ml or more after caesarian delivery. In yet an aspect of the present invention the term “postpartum haemorrhage (PPH)” is defined as a blood loss of about 1000 ml or more after vaginal delivery and about 1000 ml or more after caesarian delivery. PPH is commonly associated with uterine atony and ineffective contraction of the uterus after delivery. Other causes of PPH are trauma, retained placenta, and coagulopathy.

Uterine atony is a loss of tone in the uterine musculature. Normally, contraction of the uterine muscle compresses the vessels and reduces flow. This increases the likelihood of coagulation and prevents bleeds. Thus, lack of uterine muscle contraction can cause an acute haemorrhage. Many factors can contribute to the loss of uterine muscle tone, including but not limited to: overdistention of the uterus, multiple gestations, polyhydramnios, fetal macrosomia, dystocia including induction of labor and/or labor arrest, post term pregnancy, oxytocin augmentation of labor, grand multiparity (having given birth 5 or more times), precipitous labor (labor lasting less than 3 hours), magnesium sulfate treatment of preeclampsia, chorioamnionitis, halogenated anesthetics, and uterine leiomyomata.

The term “uterotonic agent” relates to an agent clinically used to induce myometrial contraction or greater tonicity of the uterus. Conventionally, uterotonic agents are used both to induce labor, treat labor arrest and to reduce postpartum haemorrhage. Oxytocin is a well-established uterotonic agent. In the general context uterotonic agents also extend to agents that are analogues of oxytocin or agents that indirectly may affect levels of oxytocin by promoting its secretion, such as serotonergic agents. Further non-limiting examples of uterotonic agents useful in accordance with the present invention are ergot alkaloids, prostaglandins, or analogues of prostaglandins Carbocetin is a useful analogue of oxytocin. Useful prostaglandins are exemplified by carboprost, misoprostol, dinoprostone and prostaglandin F2α analogues, while ergot alkaloids are exemplified by methylergonovine and ergometrine. The present invention also extends to methods and uses wherein more than one uterotonic agent is used.

The term “in combination” shall have the meaning of a therapy comprising a chemically modified heparin or heparan sulfate as described in accordance with the present invention and used in combination with at least one uterotonic agent that is effective in promoting or stimulating myometrial contractions of the uterus.

In one aspect of the present invention, a chemically modified heparin or heparan sulfate as herein claimed is administered as add-on therapy to a woman who has already been subjected to therapy with an uterotonic agent.

In still an aspect of the invention, a chemically modified heparin or heparan sulfate as herein defined is administered simultaneously with an uterotonic agent.

In yet an aspect of the invention, the chemically modified heparin or heparan sulfate as herein defined is administered sequentially with an uterotonic agent.

The term “treatment of PPH” relates to a therapy providing a response from the administration of the chemically modified heparin or heparan sulfates as claimed and described herein. In one aspect, the treatment of PPH according to the present invention is performed as an intervening administration therapy that following the administration initiates a process leading to the establishment of effective myometrial contractions of the uterus and treatment of PPH.

One aspect of the invention relates to prevention of PPH in women who are in a risk category. In this aspect the patient (woman) is subjected to administration directly after delivery and prior to onset of bleeding which initiates a process that leads to the establishment of effective myometrial contractions of the uterus and thereby counteracting an event of PPH. Examples of such patients are women who have been determined to or expected to suffer from uterine atony and insufficient uterine contractions, as may be the risk if a patient has been exposed to uterine overdistention, prolonged use of uterotonic agents, rapid or prolonged labor, multiparity, earlier PPH and chloroamnionitis. Especially targeted or eligible patients are those who have been subjected to uterotonic agents in order to induce labor, to treat labor arrest, or both to induce labor and to treat labor arrest, patients who are determined not to respond to the administration of an uterotonic agent, or patients treated with high doses of uterotonic agents. The preventive methods may involve subcutaneous administration or topical administration, exemplified by intravaginal administration, of a chemically modified heparin or heparan sulfate as claimed and described herein.

In the context of the present invention “labor induction” generally is defined as an intervention that directly or indirectly onsets a sufficiently effective labor from myometrial contractions of the uterus to accomplish a progress resulting in delivery and childbirth.

Labor can be induced in a number of ways, all well known to the skilled person. Examples of methods for inducing labor are physical stimulation processes; administration of oxytocin, prostaglandin E or derivatives thereof, such as misoprostol and dinoprostol; rupturing the amniotic sac; expanding the cervix, and the administration of an intracervical balloon. Also combinations of these labor inducing processes can be used.

The term “labor arrest” is used in the context of the present invention to characterize abnormalities in labor during all stages of labor starting from once the pregnant woman is having repetitive uterine contractions. Normal progress of labor is defined as regular myometrial contractions of the uterus leading to a cervical dilatation of at least about 1 cm per hour until a dilatation of 10 cm. In the context of the present invention normal progress of labor is also defined as effective labor.

Labor arrest is defined as a condition varying from a slower than normal progress (i.e. less than about 1 cm cervical dilatation during 1 hour, during 1-2 hours or during at least 2 hours) to a complete absence of progress of cervical ripening and myometrial contractions of the uterus. A woman can enter into labor arrest at different stages of labor. Early stage labor arrest (sometimes called “primary arrest”) is often due to impaired cervical dilatation and in late phase of the labor (i.e. when the woman is dilated ≧5-6 cm and with a normal progress initially cm and called “secondary arrest”) due to impaired or insufficient myometrial contractions of the uterus. The meaning of labor arrest in this context extends to clinically common terms like dystocia, slow progress in labor, arrest of labor, complete cessation of progress, dysfunctional labor failure and cephalopelvic disproportion occurring after repetitive uterine contractions have been experienced.

Sulfated glycosam inoglycans with low anticoagulant effect, such as an anti-factor Xa activity below 200 IU/mg, are disclosed herein for the treatment or prevention of PPH. The sulfated glycosam inoglycans are administered in combination with at least one uterotonic agent capable of promoting myometrial contractions of the uterus, in the treatment of PPH. The glycosam inoglycans are sulfated glycosam inoglycans selected from the group consisting of heparan sulfate, depolymerized heparan sulfate, heparin, depolymerized heparin (e.g. low molecular weight heparin) dermatan sulfate, dermatan sulfate, chondroitin sulfates and depolymerized chondroitin sulfates.

In one aspect of the invention, an effective amount of at least one chemically modified heparin or heparan sulfate with an antifactor IIa activity of less than 30 IU/mg and an antifactor Xa activity of less than 30 IU/mg, is administered to a woman suffering from PPH in combination with at least one agent capable of promoting myometrial contractions of the uterus. In still an aspect of the invention the chemically modified heparin or heparan sulfate to be used in the method of the present invention has an anti-factor Xa activity of 10 IU/mg or less and an anti-factor IIa activity of 10 IU/mg or less.

In one aspect, the chemically modified heparin or heparan sulfate administered according to be used according to the invention has an average molecular weight (Mw) of 30 000 Da or less. In another aspect the chemically modified heparin or heparan sulfate administered according to the invention has an average molecular weight (Mw) of less than 20 000 Da. In another aspect the chemically modified heparin or heparan sulfate administered according to the invention has an average molecular weight (Mw) of 10 000 Da or less. In another aspect the chemically modified heparin or heparan sulfate administered according to the invention has an average molecular weight (Mw) not higher than 8 000 Da. In yet another aspect the chemically modified heparin or heparan sulfate administered according to the invention has an average molecular weight (Mw) not higher than 7 000 Da.

The anticoagulant activity of heparin, Low Molecular Weight Heparins and other heparin derivatives is often measured as their ability to potentiate the inhibition of coagulation factor Xa and factor IIa by antithrombin. Methods for measuring anti-factor Xa- and anti-factor IIa activity are well known to the skilled person and are also described in pharmacopoeias such as the European pharmacopoeia (Pharm Eur) and the United States pharmacopoeia (USP).

The anticoagulant activity can be abrogated by for example selective periodate oxidation (see e.g. Fransson L A, and Lewis W, Relationship between anticoagulant activity of heparin and susceptibility, to periodate oxidation, FEBS Lett. 1979, 97: 119-23; Lindahl et al., Proc Natl Acad Sci USA, 1980; 77(11):6551-6555) but also by other means known to the skilled person.

In yet an aspect of the invention the disaccharide structure of the chemically modified heparin or heparan sulfate is essentially devoid of non-sulfated glucuronic and iduronic units and having an anti-factor Xa activity of 10 IU/mg or less and an anti-factor IIa activity of 10 IU/mg or less.

In yet an aspect of the invention the chemically modified heparin is a low anticoagulant heparin with an anti-factor Xa activity of 10 IU/mg or less and an average molecular weight not higher than 8 000 Da or not higher than 7 000 Da.

In one aspect, the invention is directed to the use of a chemically modified heparin; wherein the anticoagulant effect of heparin has been eradicated by treatment with periodate to eliminate antithrombin binding affinities. One non-limiting way of obtaining such a chemically modified heparin is periodate oxidation followed by alkaline β-elimination of the product. This process leads to elimination of the anticoagulant activity. The process disclosed in U.S. Pat. No. 4,990,502 (Lormeau et al) demonstrates one way of treating native heparin to selectively cleave the pentasaccharide sequences responsible for the anticoagulant effect and a following depolymerization that results in a low anticoagulant heparin with a an average molecular weight 5.8 to 7.0 kDa.

In one aspect of the invention, the chemically modified heparin for use according to the invention has an average molecular weight (Mw) from about 4.6 to about 6.9 kDa.

One aspect of the invention is a chemically modified heparin or heparan sulfate with an antifactor IIa activity of less than 10 IU/mg and an antifactor Xa activity of less than 10 IU/mg comprising;

(i) polysaccharide chains essentially free of chemically intact saccharide sequences mediating the anticoagulant effect; and
(ii) polysaccharide chains corresponding to molecular weights between 1.2 and 12 kDa with a predominantly occurring disaccharide according to (Formula I),

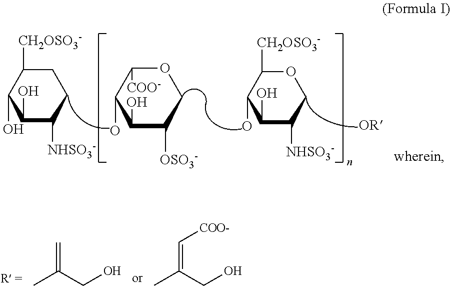

n is an integer from 2 to 20
   for use in combination with at least one uterotonic agent in the treatment or prevention of postpartum haemorrhage.

In this context, a chemically modified heparin or heparin sulfate, comprising polysaccharide chains essentially free of chemically intact saccharide sequences mediating the anticoagulant effect means that the polysaccharide chains have been treated chemically to modify essentially all the pentasaccharides specifically mediating an anticoagulant effect by antithrombin (AT).

The predominantly occurring polysaccharide chains of such a chemically modified heparin have between 6 and 12 disaccharide units with molecular weights from 3.6-7.2 kDa, while at least 70% of the polysaccharide chains have a molecular weight above at least 3 kDa. The distribution of polysaccharides and their corresponding molecular mass expressed as cumulative % of weight would be according to the table:

| Molecular mass, | Cumulative weight, |
|---|---|
| kDa | % |
| >10 | 4-15 |
| >8 | 10-25 |
| >6 | 22-45 |
| >3 | >70 |

Furthermore, the polysaccharide comprises saccharide chains having the reduced end residue as shown in Formula I and is essentially free of intact non-sulfated iduronic and/or glucuronic acids.

In one aspect, this chemically modified heparin comprises modified glucosamines present as signals in the interval of 5.0 to 6.5 ppm in a ¹H-NMR spectrum with an intensity (% ratio) of less than 4% in relation to the signal at 5.42 ppm from native heparin. These glucosamine signals may be present at 5.95 ppm and 6.15 ppm. In one aspect, less than 1% of the total content of glucosamines is modified.

In this context, “modified glucosamines” has the meaning of glucosamines having a residue structure not expected to be found in a ¹H-NMR spectrum from heparin products or low molecular weight heparin products (depolymerized heparins). The appearance of modified glucosamines may be attributed to the chemical modification process for oxidizing non-sulfated iduronic and/or glucuronic acid in order to substantially eliminate the anticoagulant effect. It is desirable to minimize the presence of modified glucosamines as they may represent unpredictable characteristics of the chemically modified heparin product, such as non-specific depolymerization.

In one aspect, the chemically modified heparin comprises modified glucosamines in the non-reducing ends with unsaturated bonds. Such modified glucosamines are present as signals at 5.95 ppm and 6.15 ppm in an ¹H-NMR spectrum.

The present invention relates to a treatment with a chemically modified heparin or heparan sulfate as described herein in combination with one or more uterotonic agents capable of promoting or stimulating myometrial contractions of the uterus administered to the woman after delivery and due to uterine atony leading to inadequate compression of the vessels. In one aspect of the invention, the uterotonic agent is oxytocin. Thus, when the chemically modified heparin or heparan sulfate is administered as an adjuvant to oxytocin it promotes the oxytocin induced myometrial contractions of the uterus. The treatment regimen will be set by the skilled treating physician or personnel and in accordance with current practice and preferably set to fit with the clinical routines for oxytocin as the chemically modified heparin or heparan sulfate will be administered in combination with oxytocin. In one aspect of the invention, the chemically modified heparin or heparan sulfate is administered up to once every hour or up to once every second hour. In one aspect of the invention, the chemically modified heparin or heparan sulfate is administered 1-24 times/24 h. In yet an aspect of the invention the chemically modified heparin or heparan sulfate is administered 12-24 times/24 h. In still yet an aspect of the invention, the chemically modified heparin or heparan sulfate is administered 1-36 times/36 h. In still yet an aspect of the invention, the chemically modified heparin or heparan sulfate is administered 18-36 times/36 h. The administration can be performed intravenously and/or subcutaneously. In one aspect the chemically modified heparin or heparan sulfate is administered by continuous infusion. Under current clinical practice oxytocin is administered intravenously.

In one aspect of the invention, the woman receives up to 360 mg of the chemically modified heparin or heparan sulfate per dose. In yet an aspect, the woman receives up to 250 mg of the chemically modified heparin or heparan sulfate per dose. In still yet an aspect, the woman receives up to 200 mg of the chemically modified heparin or heparan sulfate per dose.

In one aspect of the invention, the woman receives up to about 2.0 g of the chemically modified heparin or heparan sulfate per 24 h. In another aspect, the woman receives up to about 1.5 g of the chemically modified heparin or heparan sulfate per 24 h. As a non-limiting example, the 1.5 g is administered 6 times in doses of 250 mg.

In one aspect of the invention the dose of the uterotonic agent when oxytocin is selected may vary from 1 to 80 IU. In one aspect oxytocin is administered in a dose of up to 10 IU 4-5 times/24 h.

In one aspect, a chemically modified heparin or heparan sulfate used in accordance with the invention is included in a kit with at least a one uterotonic agent capable of promoting myometrial contractions of the uterus. The chemically modified heparin or heparan sulfate and the at least one uterotonic agent can be provided in single- or multidose forms adapted to different clinical situations. For example, the uterotonic agent or agents and the chemically modified heparin or heparan sulfate preparations can be provided in the form of a kit with prefilled syringes or ampoules comprising different doses. The skilled treating personnel or physician may select a dose in accordance with clinical practice. For example, the skilled physician or personnel treating the woman may select an initially low dose of uterotonic agent, such as from 1 to 10 IU of oxytocin together with a dose of chemically modified heparin or heparan sulfate of up to 360 mg. The preparations may include uterotonic agent and chemically modified heparin or heparan sulfate combined or separately. In the case the contractile response is inadequate, a higher dose is selected from the kit, for example with a higher concentration of oxytocin or oxytocin combined with a supplementary uterotonic agent. Preferably, the preparations suitable for acute situations are adapted for intravenous administration.

In one aspect, a chemically modified heparin or heparan sulfate used in accordance with the invention is included in a kit together with a multidose form of at least an uterotonic agent adapted for administration in several doses. In one example, the kit comprises a multidose form of oxytocin and the chemically modified heparin or heparan sulfate is administered in combination with an initial low or standardized dose of oxytocin. Should the patient remain in PPH, oxytocin may be administered one or several times with controlled doses from the multidose form, until myometrial contractions of the uterus are established.

The methods and uses according to the invention can comprise administration of the chemically modified heparin or heparan sulfates having the features as defined in any of the other parts of this specification and claims.

The chemically modified heparin or heparan sulfate used in accordance with the invention, can be administered systemically as pharmaceutical compositions by parenteral administration, such as by subcutaneous or intravenous injection. In one aspect, for acute situations the chemically modified heparin or heparan sulfate and the uterotonic agent are administered parenterally. In another aspect, when the use is preventive, the chemically modified heparin or heparan sulfate and the uterotonic agent can be administrated subcutaneously or topically. Also, several uterotonic agents may be administered together with the chemically modified heparin or heparan sulfate, as exemplified with oxytocin together with a prostaglandin. For parenteral administration the chemically modified heparin or heparan sulfate and/or the uterotonic agent(s) can be incorporated into a solution or suspension, which also contain one or more adjuvants such as sterile diluents such as water for injection, saline, fixed oils, polyethylene glycol, glycerol, propylene glycol or other synthetic solvents, antibacterial agents, antioxidants, chelating agents, buffers and agents for adjusting the osmolarity. The parenteral preparation can be delivered in ampoules, vials, disposable syringes or as infusion arrangements.

In one aspect, the present invention relates to a chemically modified heparin or heparan sulfate having an antifactor IIa activity of less than 10 IU/mg and an antifactor Xa activity of less than 10 IU/mg for use in combination with at least one uterotonic agent capable of promoting myometrial contractions of the uterus in the treatment of PPH. In yet another aspect, the present invention relates to the use of a chemically modified heparin or heparan sulfate having an antifactor IIa activity of less than 10 IU/mg and an anti-factor Xa activity of less than 10 IU/mg in combination with at least one uterotonic agent capable of promoting myometrial contractions of the uterus for the manufacture of a medicament for use in the treatment of PPH.

Encompassed by the present invention is any combination of the embodiments and aspects disclosed in the present invention.

The invention will be further disclosed in the following non-limiting examples. The processes described and exemplified in the following section include different aspects of counteracting or eliminating non-specific depolymerization.

### EXAMPLES

The following examples 1 to 9 serve as examples how to produce chemically modified heparin or heparan sulfates for use according to the present invention.

The substance is prepared from sodium heparin. The preparation involves selective oxidation of non-sulfated uronic acid residues in heparin by periodate, including the glucuronic acid moiety in the pentasaccharide sequence that binds AT. Disruption of the structure of this residue annihilates the high-affinity interaction with AT and, consequently, the anticoagulant effect (measured as a-FXa or a-FIIa) is essentially depleted. Subsequent alkaline treatment, beta-elimination reaction results in cleavage of the polymer at the sites of non-sulfated uronic acids that have been oxidized by periodate. Together, these manipulations lead to a loss of anticoagulant activity along with adequate de-polymerization of the heparin chain.

Further, the resulting reducing end terminal at the site of cleavage is reduced by NaBH₄, which converts the terminal aldehyde to the corresponding diols which are more stable. Subsequently, additives, impurities and side-products are removed by repeated precipitations with ethanol, filtration and centrifugations. Thereafter the substance is obtained in powder form by drying with vacuum and heat. The drug substance will be dissolved in a sterile aqueous buffer to yield the drug product, which is intended for intravenous or subcutaneous administration.

The processes so far described generally include the steps of oxidation, polymer cleavage (alkaline hydrolysis) and reduction. The processes according to the present invention are developed in order to counteract or eliminate any type of non-specific depolymerization of the heparin chains. Non-specific polymerization in this context means generally such depolymerization that is not related to the specific alkaline beta-elimination reaction. Non-specific depolymerization results in structural instabilities of the product that may result in further depolymerization and discoloration during storage of the purified product. In addition, it may contribute to the appearance of atypical species appearing in NMR spectra not normally found in heparin.

### Example 1

### Oxidation of Non-Sulfated Glucuronic- and Iduronic Acid (Residues), Deletion of AT-Binding Pentasaccharide and Anticoagulant Activity

A quantity of about 3000 grams of heparin is dissolved in purified water to obtain a 10-20% w/v solution. The pH of this solution is adjusted to 4.5-5.5. The sodium metaperiodate (NaIO₄) is subsequently added to the process solution; quantity of periodate 15-25% of the weight of heparin. The pH is again adjusted to 4.5-5.5. The reaction is protected from light. The process solution is reacted during the 18-24 hours with constant stirring maintenance of the temperature at 13-17° C., while the temperature is reduced to 5° C. during the last two hours.

### Termination of the Oxidation Reaction and Removal of Iodine-Containing Compounds

Ethanol (95-99.5%) is added to the reaction mixture over a period of 0.5-1 hour, with careful stirring and at a temperature of 5-25° C. The volume of ethanol to be added is in the range 1-2 volumes of ethanol per volume of process solution. The oxidized heparin is then allowed to precipitate and sediment for 15-20 hours, after which the mother liquor is decanted and discarded.

Next, the sediment is dissolved in purified water to obtain a 15-30% w/v process solution. NaCl is added to obtain a concentration of 0.15-0.30 mol/liter in the process solution. Stirring continues for another 0.5-1 hour while maintaining the temperature of 5-25° C. Subsequently 1.0-2.0 volumes of ethanol (95-99.5%) per volume of process solution are added to this solution with stirring, during a period of 0.5-1 hour. This precipitates the product from the solution.

### De-Polymerization of Polysaccharide Chains by an Alkaline Beta Elimination Process

After the mother liquor has been decanted and discarded, the sediment is stirred in approximately 7 litres of water until completely dissolved, the concentration of the solution is now 15-30%. While maintaining the temperature at 5-25° C. a 4 M NaOH solution is added slowly until a pH of 10.5-12 is obtained. The reaction is initiated and proceeds for 15-95 minutes. At this time, the pH of the solution is recorded and 4 M HCl is added slowly until a pH of 5.5-7 is obtained.

### Reduction of Reducing End Terminals

While maintaining the temperature at 13-17° C., the pH of the solution is adjusted to 5.5-6.5. A quantity of 130-150 grams of sodium borohydride is then added to the solution while the pH will increase to 10-11, the reaction is continued for 14-20 hours. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution.

The purification continues according to example 5

### Example 2

### Oxidation of Glucuronic and Iduronic Acid (Residues), Deletion of Anticoagulant Activity

A quantity of about 3000 grams of heparin is dissolved in purified water to obtain a 10-20% w/v solution. The pH of this solution is adjusted to 4.5-5.5. The sodium metaperiodate (NaIO₄) is subsequently added to the process solution; quantity of periodate 15-25% of the weight of heparin. The pH is again adjusted to 4.5-5.5. The reaction is protected from light. The process solution is reacted during the 22-26 hours with constant stirring and maintenance of the temperature at 13-17° C., while the temperature is reduced to 5° C. during the last two hours. The pH at the end of the reaction period is measured and recorded.

### Termination of the Oxidation Reaction and Removal of Iodine-Containing Compounds

Ethanol (95-99.5%) is added to the reaction mixture over a period of 0.5-1 hour, with careful stirring and at a temperature of 5-25° C. The volume of ethanol to be added is in the range 1-2 volumes of ethanol per volume of process solution. The oxidized heparin is then allowed to precipitate and sediment for 15-20 hours, after which the mother liquor is decanted and discarded.

### De-Polymerization of Polysaccharide Chains by an Alkaline Beta Elimination Process

After the mother liquor has been decanted and discarded, the sediment is stirred in approximately 7 litres of water until it appears visually to be completely dissolved. While maintaining the temperature at 20-25° C. 4 M NaOH is added slowly until a pH of 10.5-12 is obtained and the reaction thus initiated is allowed to proceed for 15-95 minutes. At this time, the pH of the solution is recorded and 4 M HCl is added slowly until a pH of 5.5-7 is obtained.

### Reduction of Reducing End Terminals

After the mother liquor has been decanted and discarded, the sediment is dissolved by addition of purified water until a concentration of the process solution of 15-30% w/v is obtained. While maintaining the temperature at 13-17° C., the pH of the solution is adjusted to 5.5-6.5. A quantity of 130-150 grams of sodium borohydride is then added to the solution and dissolved, the pH will immediately increase to a pH of 10-11, the reaction is continued for 14-20 hours. The pH of the solution, both prior to and after this reaction period, is recorded. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution.

Purification continues according to Example 5.

### Example 3

### Oxidation of Glucuronic and Iduronic Acid (Residues), Deletion of Anticoagulant Activity

A quantity of about 3000 grams of heparin is dissolved in purified water to obtain a 10-20% w/v solution. The pH of this solution is adjusted to 4.5-5.5. The sodium metaperiodate (NaIO₄) is subsequently added to the process solution, quantity of periodate 15-25% of the weight of heparin. The pH is again adjusted to 4.5-5.5. The reactor is protected from light. The process solution is reacted during the 18-24 hours with constant stirring maintenance of the temperature at 13-17° C., while the temperature is reduced to 5° C. during the last two hours.

### De-Polymerization of Polysaccharide Chains by an Alkaline Beta Elimination Process

While maintaining the temperature at 5-25° C., 4 M NaOH solution is added slowly until a pH of 10.5-12 is obtained. The reaction is initiated and proceeds for 15-95 minutes. At this time, the pH of the solution is recorded and 4 M HCl is added slowly until a pH of 5.5-7 is obtained.

### Reduction of Reducing End Terminals

While maintaining the temperature at 13-17° C., the pH of the solution is adjusted to 5.5-6.5. A quantity of 130-200 grams of sodium borohydride is then added to the solution while the pH will increase to 10-11, the reaction is continued for 14-20 hours. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution.

### Precipitation of Reduced Product and Initial Removal of Iodine-Containing Compounds

Ethanol (95-99.5%) is added to the reaction mixture over a period of 0.5-1 hour, with careful stirring and at a temperature of 5-25° C. The volume of ethanol to be added is in the range 1-2 volumes of ethanol per volume of process solution. The oxidized heparin is then allowed to precipitate and sediment for 15-20 hours, after which the mother liquor is decanted and discarded.

Next, the sediment is dissolved in purified water to obtain a 15-30% w/v process solution. NaCl is added to obtain a concentration of 0.15-0.30 mol/liter in the process solution

Purification continues according to Example 5.

### Example 4

### Oxidation of Glucuronic and Iduronic Acid (Residues), Deletion of Anticoagulant Activity

A quantity of about 3000 grams of heparin is dissolved in purified water to obtain a 10-20% w/v solution. The pH of this solution is adjusted to 4.5-5.5. The sodium metaperiodate (NaIO₄) is subsequently added to the process solution, quantity of periodate 15-25% of the weight of heparin. The pH is again adjusted to 4.5-5.5. The reactor is protected from light. The process solution is reacted during the 18-24 hours with constant stirring maintenance of the temperature at 13-17° C., while the temperature is reduced to 5° C. during the last two hours. Next, glycerol is added to quench the reaction, i.e. to convert residual periodate to iodate, 150-200 ml of a 85% glycerol solution is added and reacted for 30-60 minutes while stirring.

### Precipitation of Product Removal of Iodine-Containing Compounds and Quencher/Reaction Products

Ethanol (95-99.5%) is added to the reaction mixture over a period of 0.5-1 hour, with careful stirring and at a temperature of 5-25° C. The volume of ethanol to be added is in the range 1-2 volumes of ethanol per volume of process solution. The oxidized heparin is then allowed to precipitate and sediment for 15-20 hours, after which the mother liquor is decanted and discarded.

Next, the sediment is dissolved in purified water to obtain a 15-30% w/v process solution. NaCl is added to obtain a concentration of 0.15-0.30 mol/liter in the process solution. Stirring continues for another 0.5-1 hour while maintaining the temperature of 5-25° C. Subsequently 1.0-2.0 volumes of ethanol (95-99.5%) per volume of process solution are added to this solution with stirring, during a period of 0.5-1 hour. This precipitates the product from the solution.

### De-Polymerization of Polysaccharide Chains by an Alkaline Beta Elimination Process

After the mother liquor has been decanted and discarded, the sediment is stirred in approximately 7 litres of water until it appears visually to be completely dissolved. While maintaining the temperature at 5-25° C. 4 M NaOH is added slowly until a pH of 10.5-12 is obtained and the reaction thus initiated is allowed to proceed for 60-95 minutes. At this time, the pH of the solution is recorded and 4 M HCl is added slowly until a pH of 5.5-7 is obtained.

### Reduction of Reducing End Terminals

After the mother liquor has been decanted and discarded, the sediment is dissolved by addition of purified water until a concentration of the process solution of 15-30% w/v is obtained. While maintaining the temperature at 13-17° C., the pH of the solution is adjusted to 5.5-6.5. A quantity of 130-150 grams of sodium borohydride is then added to the solution and dissolved, the pH will immediately increase to a pH of 10-11, the reaction is continued for 14-20 hours. The pH of the solution, both prior to and after this reaction period, is recorded. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution.

Purification proceeds according to Example 5.

### Example 5

### Purification of the Product

### Removal of Process Additives and Impurities, Addition of Counter-Ions and Filtration

Process solutions according to Examples 1-4 arriving from the final chemical modification step of reducing the end terminals by borohydride is worked up according the methodologies outlined below.

One volume of process solution is then added to 1.5-2.5 volumes of ethanol (95-99.5%) followed by centrifugation at >2000 G, at <20° C. for 20-30 minutes, after which the supernatant is decanted and discarded.

The product paste obtained by centrifugation is then dissolved in purified water to obtain a product concentration 10-20% w/v. Then NaCl is added to obtain a concentration of 0.20-0.35 mol/liter. Next 1.5-2.5 volumes of ethanol (95-99.5%) are added per volume of process solution which precipitates the product from the solution. Centrifugation follows as described above.

Next the remaining paste is added purified water to dissolve. The product concentration would now be in the range of 10-20% w/v. The pH of the product solution is now adjusted to 6.5-7.5. The solution is then filtered to remove any particulates. Then, to one volume of process solution is added 1.5-2.5 volumes of ethanol (95-99.5%). Centrifugation follows at >2000 G, and at <20° C. for 20-30 minutes after which the supernatant is decanted and discarded.

### Dewatering of Precipitate Paste and Reduction of Particle Size.

A reactor is filled with ethanol, volume about 2 liters. While stirring the ethanol, the precipitate paste is added. The mechanical stirring solidifies the paste and replaces the water present by the ethanol giving a homogenous particle suspension. The stirring is discontinued after 1-2 hours after which the particles are allowed to sediment. After removal of excessive liquid, the particles are passed through a sieve or a mill to obtain smaller and uniform sized particles.

### Drying of Product

The product is distributed evenly onto trays, and placed in a vacuum cabinet. Vacuum is applied and heating is performed at 35-40° C. A stream of nitrogen is passed through the drier at this time while maintaining the low pressure in the dryer. When a constant weight is obtained of the product, i.e. no further evaporation is noticed, the drying is considered complete. The product is packed and protected from humidity.

### Example 6

### Oxidation of Glucuronic and Iduronic Acid (Residues), Deletion of Anticoagulant Activity

A quantity of about 3000 grams of heparin is dissolved in purified water to obtain a 10-20% w/v solution. The pH of this solution is adjusted to 4.5-5.5. The sodium metaperiodate (NaIO₄) is subsequently added to the process solution, quantity of periodate 15-25% of the weight of heparin. The pH is again adjusted to 4.5-5.5. The reaction is protected from light. The process solution is reacted during the 18-24 hours with constant stirring maintenance of the temperature at 13-17° C., while the temperature is reduced to 5° C. during the last two hours.

### De-Polymerization of Polysaccharide Chains by an Alkaline Beta Elimination Process

While maintaining the temperature at 5-25° C. 4 M NaOH is added slowly until a pH of 10.5-12 is obtained and the reaction thus initiated is allowed to proceed for 15-95 minutes. At this time, the pH of the solution is recorded and 4 M HCl is added slowly until a pH of 5.5-7 is obtained.

### Reduction of Reducing End Terminals

After the mother liquor has been decanted and discarded, the sediment is dissolved by addition of purified water until a concentration of the process solution of 15-30% w/v is obtained. While maintaining the temperature at 13-17° C., the pH of the solution is adjusted to 5.5-6.5. A quantity of 130-200 grams of sodium borohydride is then added to the solution and dissolved, the pH will immediately increase to a pH of 10-11, the reaction is continued for 14-20 hours. The pH of the solution, both prior to and after this reaction period, is recorded. After this reaction time, a dilute acid is added slowly in order to adjust the pH to a value of 4, this degrades remaining sodium borohydride. After maintaining a pH of 4 for 45-60 minutes, the pH of the solution is adjusted to 7 with a dilute NaOH solution. Purified water is now added to the solution until a conductivity of 15-20 m S/cm is obtained of the reaction solution.

### Purification of Product by Anion Exchange Chromatography

A column with a diameter 500 mm is packed with media, DEAE-Sepharose or QAE-Sepharose to a volume of 25-30 liters corresponding to a bed height of 10-15 cm. The chromatography is performed in 3-4 cycles to consume the entire product.

Next buffers are prepared,

Equilibration buffer, Buffer A, 15 mM phosphate, 150 mM NaCl
Elution buffer, Buffer B, 2 M NaCl solution
Sanitation buffer, 0.5 M NaOH

The chromatography step is performed at 15-25° C., at flow rate of ≦200 cm/hour or approx. 350 liters/hour.

The column is equilibrated with the equilibration buffer until the eluent has a conductivity of 15-20 m S/cm. Next the oxidized heparin solution is pumped into the column. The quantity of crude product to be applied corresponds to <40 g/liter of chromatography media.

An isocratic wash follows with equilibration buffer and is discontinued when the UV 210-254 nm has reached a baseline. Typically 5 bed volumes of buffer are required to reach baseline. Chemicals added to the process and products formed of these are removed.

Next, the ionic strength of the buffer applied onto the column is linearly increased by performing a gradient elution. The Buffer A decreases from 100% to 0% replaced by 100% Buffer B over 5 bed volumes. The product, eluate is collected when the UV absorbance is >0.1 AU and is discontinued when the signal is <0.1 AU. Sanitation of the column is then performed after which it is again prepared for the next cycle of chromatography. Eluates from all runs are combined and stored at 15-25° C.

### De-Salting of the Product

One volume of the combined eluates from previous step is added 3 volumes of 95-99.5% ethanol, 15-25° C., under constant stirring. This precipitates the product out of solution. The product is allowed to sediment for >3 hours. Next, the sediment is dissolved in purified water to a concentration of 15-25%. The solution is now added to cold ethanol (<−5° C.) 95-99.5%, typically 5 volumes of ethanol per one volume of product solution are consumed. Next follows centrifugation in a continuous mode, >2000 G, the product paste is thereafter collected and prepared for drying.

### Drying of Product

The product is distributed evenly onto trays, and placed in a vacuum cabinet. Vacuum is applied and heating is performed at 35-40° C. A stream of nitrogen is passed through the drier at this time while maintaining the low pressure in the dryer. When a constant weight is obtained of the product, i.e. no further evaporation is noticed, the drying is considered complete. The product is milled and made homogenous, thereafter packed and protected from humidity.

### Example 8

Low anticoagulant heparin produced according to the examples 1 and 3 was subjected to 1H-NMR analysis and compared to the spectrum of native heparin.

Table II demonstrates signals in the interval 5.00 ppm to 6.50 ppm not present in native heparin generated from non-reducing end unsaturated glucosamines. The results of Table II show that it is possible to reduce the presence of such compounds not predicted to be present in spectrum from native heparin to low levels. In comparison, the current limit applicable to heparin quality control, monograph 7, EDQM is <4% compared to the signal at 5.42 ppm for any signal in the region 5.70-8.00 ppm.

TABLE II
Qualitative results of a low anticoagulant heparin
with regards to unusual signals. Signal intensity
for signals 6.15 and 5.95 ppm in a 1H-NMR spectra

Intensity (% ratio) to 5.42 ppm
signal of a native heparin
following EDQM, monograph 7

| | Production | 6.15 ppm | 5.95 ppm |
|---|---|---|---|
| Sample | method | % of ref. signal | % of ref. signal |
| Batch 1 | Example 1 | 11 | 12 |
| Batch 2 | Example 1 | 13 | 16 |
| Batch 3 | Example 3 | 2 | 2 |

Further, the presence of non reducing end unsaturated glucosamines was also quantified by combined 1H-NMR and 13C-NMR spectra evaluation (HSQC) and demonstrated as mol % of total glucosamines (see Table III).

Furthermore, the sample was analyzed by following the NMR two-dimensional (2D) method involving the combined use of proton and carbon NMR spectroscopy (HSQC) as previously described (see Guerrini M., Naggi A., Guglieri S, Santarsiero R, Torri G. Anal Biochem 2005; 337, 35-47.)

Table III demonstrates the fraction (%) of modified glucosamines compared to the total amount of glucosamines of the low anticoagulant heparin as present as signals at 5.95 ppm and 6.15 ppm in the ¹H-NMR spectrum.

TABLE III
Results from quantitative determination of unusual
signals 5.95 ppm, 6.15 ppm of total glucosamine

| | | 6.15 ppm signal | 5.95 ppm signal |
|---|---|---|---|
| | Production | mol % of | mol % of |
| Sample | method | glucosamine | glucosamine |
| Batch 1 | Example 1 | 6 | 3 |
| Batch 2 | Example 3 | <1 | <1 |

### Example 9

The product manufactured according to any one of the examples above can prepared as drug product by a conventional aseptic process, such as solution comprising 150 mg/mL of active product and Na phosphate to 15 mM, pH 6-8. The so obtained drug product is intended primarily for subcutaneous administration but suitable for intra-venous administration.

The resulting product is a depolymerized form of heparin with a projected average molecular weight of 4.6-6.9 kDa and with essentially no anticoagulant activity.

The product has a size distribution of polysaccharide polymers, with a range for n of 2-20 corresponding to molecular weights of 1.2-15 kDa. The predominant size is 6-16 disaccharide units corresponding to molecular weights of 3.6-9.6 kDa.

The molecular weight was determined by GPC-HPLC carried out with a TSK 2000 and TSK 3000 SW columns in series. Refractive index was used for evaluation. First international calibrant for LMWH was used.

Below is presented the molecular mass distribution and the corresponding part of the cumulative percentage of total weight.

TABLE IV
Distribution of polysaccharides and their corresponding
molecular mass in as cumulative % of weight for several batches

| Molecular mass, | Cumulative weight, |
|---|---|
| kDa | % |
| >15 | <1 |
| >10 | 4-15 |
| >9 | 7-20 |
| >8 | 10-27 |
| >7 | 15-35 |
| >6 | 22-45 |
| >5 | 34-56 |
| >4 | 47-70 |
| >3 | >70 |
| >2 | >85 |

The corresponding value for weight average molecular weight, Mw falls in the range 4.6-6.9 kDa

### Example 10

The stability of the drug substance (powder) and drug product dissolved in aqueous phosphate buffered solution of a chemically modified heparin produced in accordance with Examples 1 to 3 and formulated in accordance with Example 9 was studied for stability over 36 months at ambient temperature. The initial product was clear white to slight yellow solution had an absorbance at 400 nm (10% w/v solution) of 0.14, a pH of 7.0 and osmolality of 658 mOsm/kg, an average molecular weight of 5.6 kDa and a content of 150 mg/ml.

After 36 months, the drug product had the same visual appearance, an absorbance at 400 nm (10% w/v solution) of 0.13, a pH of 7.1 and osmolality of 657 mOsm/kg, an average molecular weight of 5.4 kDa and a content of 153 mg/ml.

### Example 11

### Subcutaneous Administration

Chemically modified heparin produced by the method disclosed in example 1 was labeled with tritium and administered to Sprauge Dawley rats and dogs.

### Results:

Following subcutaneous administration at 2, 8 and 24 mg heparin/kg/day in the rat and 3, 15 and 45 mg heparin/kg/day in the dog, absorption was rapid and maximal plasma levels were generally reached within 0.5 and 1.5 h in the rat and dog, respectively. The subcutaneous bioavailability was around 90% in both the rat and the dog. Interestingly, the corresponding bioavailability for heparin is about 10%.

### Example 12

### Treatment with DF01 During Late Pregnancy

### Study Design

This was a randomized, double blind, placebo-controlled, multicentre study to assess the safety and efficacy of pre-treatment with DF01 during late pregnancy in reducing labor time. Eighteen study centers in Sweden participated in the study.

DF01 is a chemically modified heparin according to the invention that is low-anticoagulant heparin chemically generated by periodate oxidation of heparin from pig intestinal mucosa, followed by β-elimination of the product following Examples 1 and 9.

The protocol stated that each subject would come to the clinic daily from the treatment start at a gestational age from week 38+0 up to week 40+0 until labor to receive a s.c. injection of the investigational medicinal product. The anticipated duration of participation in the study was 1-28 days (+screening and follow-up periods) for each subject. All women had to be induced into at the latest at 42+0 weeks of gestation. A maximum of 28 days of treatment [maximally 28 doses of the investigational medicinal product (IMP)] was given. A follow-up visit was to take place at 8-16 weeks after delivery.

### Treatments

DF01 and matching placebo, were provided as solutions for subcutaneous injection.

The pharmaceutical preparation of DF01 is a solution for subcutaneous injection, 8 mL dispensed in glass vials sealed with a rubber stopper and covered with a tear-off aluminum cap.

Each mL of the DF01 solution contains the following:
DF01, 150 mg
Phosphate buffer, 0.015 M
Benzyl alcohol, 14 mg.

A sterile physiological sodium chloride solution preserved with benzyl alcohol was used as placebo. Eight (8) mL of the placebo were provided in vials in the same way as for the drug product.

Each mL of the placebo solution contains the following:
Sodium chloride, 9 mg
Benzyl alcohol, 14 mg.

The subjects received 60 mg/day of DF01 (0.4 mL) (corresponding to 1.00 mg/kg/day in a 60 kg subject) or placebo (0.4 mL).

The products was administered by daily subcutaneous injections with treatment start at gestational age of week 38+0 to week 40+0 and treatment duration until labor. If still undelivered at 42+0 labor was to be induced. The maximum duration of treatment was 28 days. The allowed time interval between the daily injections was 24+/−6 hours, i.e. 18-30 hours. If the time limits were occasionally not met or a dose missed, the treatment could still continue.

### Results

A total of 252 women were included in the study. Out of these women, 84 received oxytocin and placebo and 94 received oxytocin and DF01.

TABLE V
All deliveries wherein oxytocin were
administered (incl. caesarians)

| Treatment | Placebo (84) | | | DF01 (94) | | |
|---|---|---|---|---|---|---|
| Bleeding | 0- | 1000- | 2000- | 0- | 1000- | 2000- |
| volume (ml) | 1000 | 2000 | 3000 | 1000 | 2000 | 3000 |
| after delivery | | | | | | |
| No of women | 71 | 11 | 2 | 87 | 6 | 1 |
| % of total | 85 | 13 | 2 | 93 | 6 | 1 |
| placebo and | | | | | | |
| DF01 | | | | | | |
| respectively | | | | | | |

TABLE VI
All deliveries wherein oxytocin were
administered (excl. caesarians)

| Treatment | Placebo | | | DF01 | | |
|---|---|---|---|---|---|---|
| Bleeding | 0- | 1000- | 2000- | 0- | 1000- | 2000- |
| volume (ml) | 1000 | 2000 | 3000 | 1000 | 2000 | 3000 |
| after delivery | | | | | | |
| No of women | 56 | 8 | 2 | 76 | 5 | 1 |
| % of total | 85 | 12 | 3 | 93 | 6 | 1 |
| placebo and | | | | | | |
| DF01 | | | | | | |
| respectively | | | | | | |

As can be seen in Tables V and VI, women receiving DF01 in combination with oxytocin bleed less after delivery compared to women who received oxytocin and placebo. Out of the women who received DF01 in combination with oxytocin, 93% did not experience PPH compared to 85% of the placebo group. 15% (13% 1000-2000 ml and 2% 2000-3000 ml) of the placebo treated women experienced PPH while the corresponding figure among the DF01 treated women was only 7% (6% 1000-2000 ml and 1% 2000-3000 ml). Excluding the women wherein the child was delivered by caesarian section gives the same result (see Table VI).

### Example 13

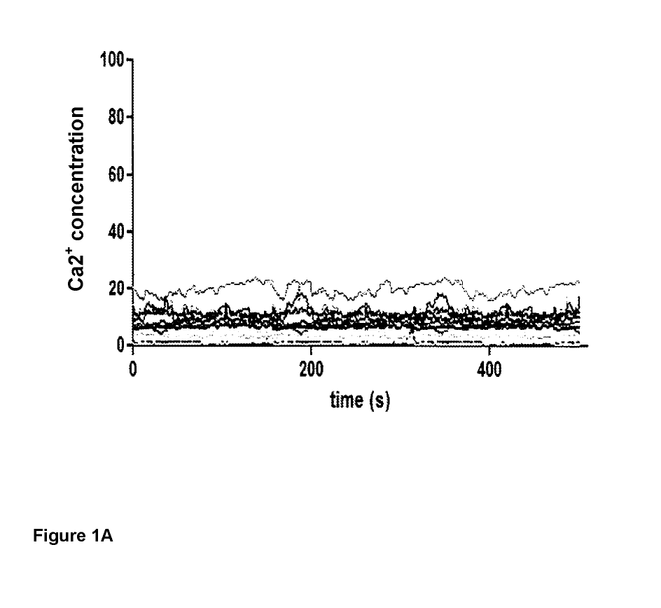

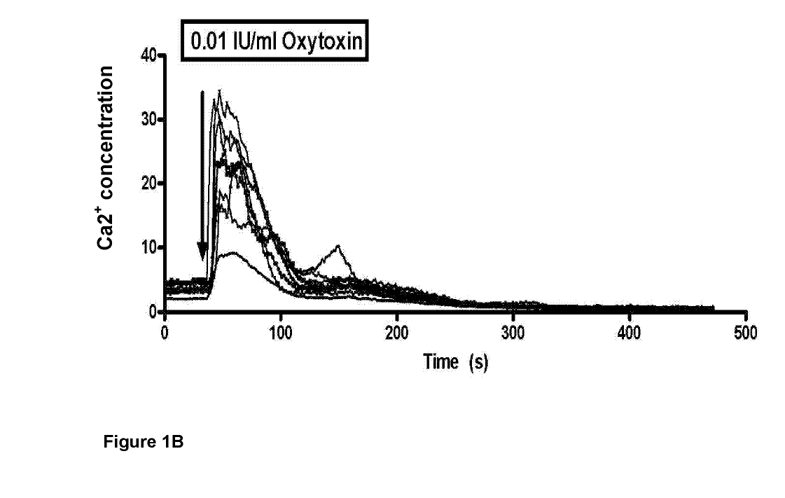

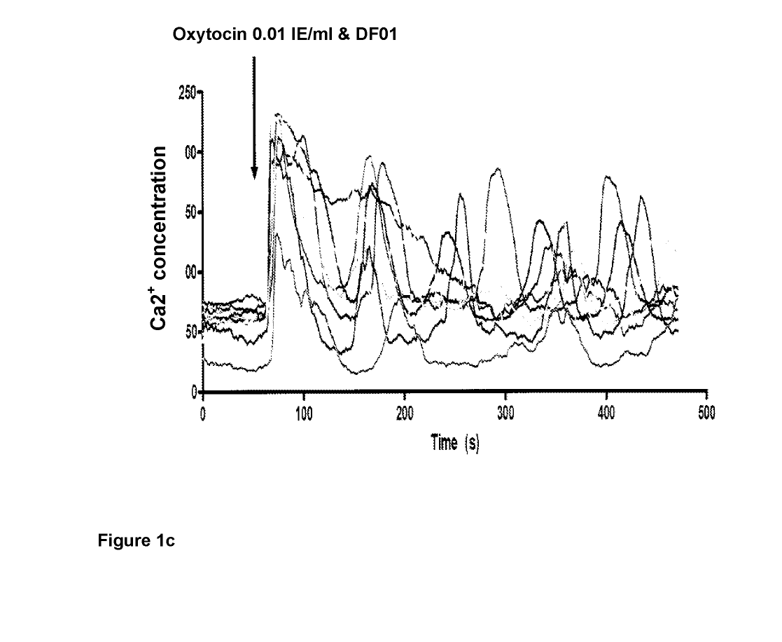

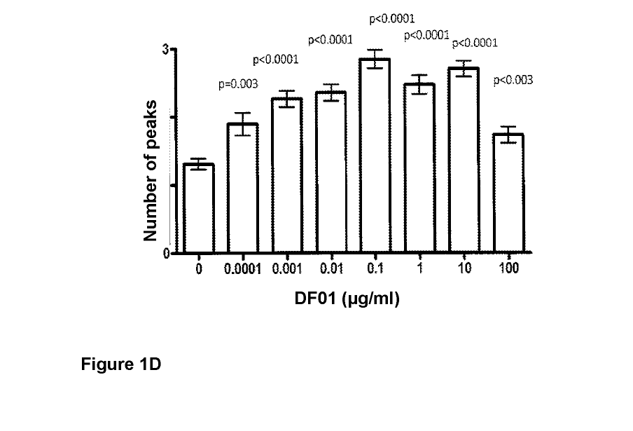

Human uterine smooth muscle cells were established in a culture. Intracellular Ca²⁺ was measured with the calcium indicator dye Fluo-4 and live cell imaging with confocal microscopy was established for the cells. The cells were treated with oxytocin and a Ca²⁺-influx to the cytosol was demonstrated (FIG. 1B).

The effect was dose-dependent with a maximum effect already at 0.05 IU/ml oxytocin. For the experiments DF01 as described Example 1 was used.

FIG. 1A shows that DF01 alone did not affect the Ca²⁺-concentration. However, when DF01 was given together with oxytocin, an increased and sustained Ca²⁺-level was attained compared oxytocin alone (FIGS. 1B and C). The dose response pattern, see FIG. 1D, shows that the number of Ca²⁺-peaks correlate with the concentration of DF01. The results demonstrate a mechanism for how DF01 exert an effect on uterine contraction by promoting and sustaining the effect of oxytocin. The mechanism was further investigated by preincubating uterine smooth muscle cells with 10 μM of verapam il for 30 m in. Verapam il did not affect the Ca²⁺ influx, induced by either oxytocin or by the combination of oxytocin and DF01. It can therefore be concluded s that L-channels not are involved.

It was further investigated if the main transport mechanism of inositol-3 phosphate (IP3) stimulated Ca²⁺ transport of the endoplasmatic reticulum. To study this pathway, 2-Aminoethoxydiphenyl borate (2-APB) was tested on Ca²⁺ after 30 min of incubation with a concentration of 100 μM. This inhibitor decreased strongly both the oxytocin and the oxytocin/DF01 stimulated Ca²⁺-transport.

To further characterize the interaction between oxytocin and DF01 the effect of the oxytocin receptor antagonist Atosiban was used and the cells subjected to the DF01 enhanced oxytocin effect on Ca²⁺ transport. Atosiban in a concentration of 10⁻⁶ M clearly inhibited the effect of both oxytocin and the combination oxytocin/DF01

The results indicate that DF01 does not by itself affect Ca²⁺-transport. However in combination with oxytocin a clear dose response enhanced stimulation of Ca²⁺ transport is noted. DF01 stabilizes the effect of oxytocin resulting in longer periods of stimulation. The effect of does not involve L-channels but rather involves IP3 stimulated Ca²⁺ influx in oxytocin signaling. The effect of the oxytocin antagonist suggests that the effect on DF01 operates on the oxytocin receptor level.

The influx of Ca within myometrial cells is directly associated with the force of the contractile activity of the myometrium (Arrowsmith et al., PLOSone, 2012, vol. 7, p. 1-11). It is therefore concluded that DF01 and chemically modified heparins according to the invention are useful agents to administer for improving myometrial contractions and to treat complications associated with inadequate or absent myometrial contractions of the uterus, such as uterine atony in PPH. In summary, DF01 and similar chemically modified heparin and heparin sulfates are regarded to be effective intervening treatments required to treat or prevent PPH by establishing effective myometrial contractions of the uterus.

Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims.

1. A kit comprising:
a chemically modified heparin or heparan sulfate in combination with at least one uterotonic agent, the chemically modified heparin or heparan sulfate having an antifactor IIa activity of less than 10 IU/mg, an antifactor Xa activity of less than 10 IU/mg, and an average molecular weight (Mw) from about 4.6 to about 6.9 kDa, and comprising
(i) polysaccharide chains essentially free of chemically intact saccharide sequences mediating the anticoagulant effect; and
(ii) polysaccharide chains corresponding to molecular weights between 1.2 and 12 kDa with a predominantly occurring disaccharide according to (Formula I), 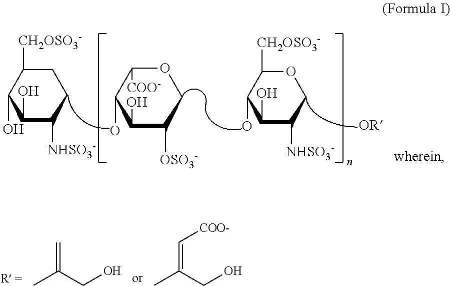
n is an integer from 2 to 20,
wherein the chemically modified heparin or heparan sulfate comprises non-reducing end unsaturated glucosamines that are presented as signals in the interval of 5.0 to 6.5 ppm in a ¹H-NMR spectrum with an intensity (% ratio) of less than 4% in relation to the signal at 5.42 ppm from native heparin.

2. The method according to claim 24, wherein the PPH appears in a woman suffering from uterine atony.

3. The method according to claim 24, wherein the PPH appears in a woman who has been induced into labor.

4. The method according to claim 24, wherein the PPH appears in a woman who has experienced labor arrest.

5. The kit according to claim 1, wherein the uterotonic agent is selected from the group consisting of oxytocin or an analogue of oxytocin, an ergot alkaloid, and a prostaglandin or an analogue of prostaglandin.

6. The kit according to claim 5, wherein the uterotonic agent is oxytocin or an analogue of oxytocin.

7. The kit according to claim 6, wherein the uterotonic agent is oxytocin.

8. The kit according to claim 6, wherein the analogue of oxytocin is carbocetin.

9. The kit according to claim 5, wherein the uterotonic agent is a prostaglandin or an analogue of prostaglandin.

10. The kit according to claim 5, wherein the uterotonic agent is an ergot alkaloid.

11. (canceled)

12. The kit according to claim 1, wherein the predominantly occurring polysaccharide chains have between 6 and 12 disaccharide units with molecular weights from 3.6 to 7.2 kDa.

13. The kit according to claim 1, wherein at least 70% of the polysaccharide chains have a molecular weight above at least 3 kDa.

14. The kit according to claim 1, wherein the chemically modified heparin or heparan sulfate has having a distribution of polysaccharides and their corresponding molecular mass expressed as cumulative % of weight according to the table:

| Molecular mass, | Cumulative weight, |
|---|---|
| kDa | % |
| >10 | 4-15 |
| >8 | 10-25 |
| >6 | 22-45 |
| >3 | >70 |

15-16. (canceled)

17. The kit according to claim 1, wherein the non-reducing end unsaturated glucosamine signals are present at 5.95 ppm and 6.15 ppm in the ¹H-NMR spectrum.

18. The kit according to claim 1, wherein the non-reducing end unsaturated glucosamines comprise less than 1% of the total content of glucosamines.

19-20. (canceled)

21. The kit according to claim 1, wherein the chemically modified heparin or heparan sulfate is essentially free of intact non-sulfated iduronic and/or glucuronic acids.

22. The method according to claim 24, wherein the chemically modified heparin or heparan sulfate is administered in a parenteral pharmaceutical preparation.

23. A pharmaceutical composition comprising:
at least one uterotonic agent; and
a chemically modified heparin or heparan sulfate having an antifactor IIa activity of less than 10 IU/mg, an antifactor Xa activity of less than 10 IU/mg, and an average molecular weight (Mw) from about 4.6 to about 6.9 kDa, and comprising
(i) polysaccharide chains essentially free of chemically intact saccharide sequences mediating the anticoagulant effect; and
(ii) polysaccharide chains corresponding to molecular weights between 1.2 and 12 kDa with a predominantly occurring disaccharide according to (Formula I), 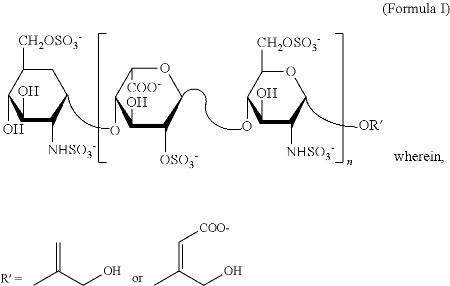
n is an integer from 2 to 20,
wherein the chemically modified heparin or heparan sulfate comprises non-reducing end unsaturated glucosamines that are presented as signals in the interval of 5.0 to 6.5 ppm in a ¹H-NMR spectrum with an intensity (% ratio) of less than 4% in relation to the signal at 5.42 ppm from native heparin.

24. A method of treating postpartum haemorrhage (PPH) comprising:
parenterally administering to a patient exhibiting PPH a chemically modified heparin or heparan sulfate having an antifactor IIa activity of less than 10 IU/mg, an antifactor Xa activity of less than 10 IU/mg, and an average molecular weight (Mw) from about 4.6 to about 6.9 kDa, and comprising
(i) polysaccharide chains essentially free of chemically intact saccharide sequences mediating the anticoagulant effect; and
(ii) polysaccharide chains corresponding to molecular weights between 1.2 and 12 kDa with a predominantly occurring disaccharide according to (Formula I), 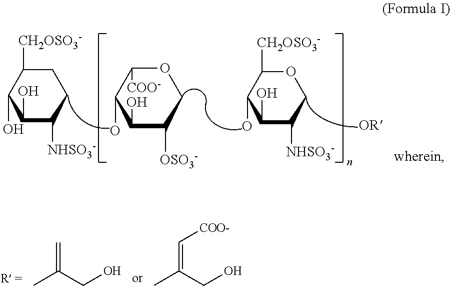
n is an integer from 2 to 20,
wherein the chemically modified heparin or heparan sulfate comprises non-reducing end unsaturated glucosamines that are presented as signals in the interval of 5.0 to 6.5 ppm in a ¹H-NMR spectrum with an intensity (% ratio) of less than 4% in relation to the signal at 5.42 ppm from native heparin; and
administering to the patient at least one uterotonic agent.

25. (canceled)

26. The method according to claim 24, wherein said parentally administering the chemically modified heparin or heparan sulfate is subsequent to said administering the at least one uterotonic agent.
